(19) United States
(12) Patent Application Publication
(10) Pub. No.: US 2004/0175691 A1
(43) Pub. Date: Sep. 9, 2004

(54) Use of the KCNQ2 and KCNQ3 genes for the discovery of agents useful in the treatment of neurological disorders

(76) Inventors: Barry S. Brown, Wilmington, DE (US); David Mckinnon, East Setauket, NY (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/820,307

(22) Filed: Apr. 8, 2004

Related U.S. Application Data
(62) Division of application No. 09/454,868, filed on Dec. 3, 1999.
(60) Provisional application No. 60/110,804, filed on Dec. 3, 1998.

(51) Int. Cl. C12Q 1/00; G01N 33/53; G01N 33/567

(52) U.S. Cl. 435/4; 435/7.2

(57) ABSTRACT

This invention relates to the co-expression of KCNQ2 and KCNQ3 genes in an appropriate mammalian cell line (e.g., HEK 293E) to provide a preparation which could be used as a high-throughput screen for the discovery of agents that are either agonists or antagonists of the expressed potassium channel. Mutations in the voltage-gated potassium channel genes, KCNQ2 and KCNQ3, have been linked to inherited forms of epilepsy in humans. One or both of these genes are believed to encode the molecular identity of the M channel. Agonists of the M channel may be effective in the treatment of epilepsy, anxiety, insomnia or other hyperexcitability disorders whereas antagonists may be effective in the treatment of Alzheimer's disease, peripheral neuropathy or other neurodegenerative diseases.

### FIELD OF THE INVENTION

This invention relates to the co-expression of KCNQ2 and KCNQ3 genes in an appropriate mammalian cell line (e.g., HEK 293E) to provide a preparation which could be used as a high-throughput screen for the discovery of agents that are either agonists or antagonists of the expressed potassium channel. Mutations in the voltage-gated potassium channel genes, KCNQ2 and KCNQ3, have been linked to inherited forms of epilepsy in humans. One or both of these genes are believed to encode the molecular identity of the M channel. Agonists of the M channel may be effective in the treatment of epilepsy, anxiety, insomnia or other hyperexcitability disorders whereas antagonists may be effective in the treatment of Alzheimer's disease, peripheral neuropathy or other neurodegenerative diseases.

### BACKGROUND OF THE INVENTION

Several neurological diseases are known to involve deficiencies in CNS neurotransmitter systems. Accordingly, cholinesterase inhibitors are used to alleviate the cholinergic deficit found in Alzheimer's disease, L-DOPA is used to supply dopamine precursor for the treatment of Parkinson's disease, and monoamine reuptake blockers are used restore the noradrenergic and serotonergic deficits associated with depression. Another approach to the treatment of these diseases is to enhance the release of the deficient neurotransmitter or to mimic its action.

Linopirdine (3,3-bis(4-pyridinylmethyl)-1-phenylindolin-2-one), disclosed in U.S. Pat. No. 5,173,489 (which is hereby incorporated by reference), has been shown to enhance K⁺-stimulated release of acetylcholine, dopamine and glutamate in the mammalian CNS. It has been reported that linopirdine, when tested for electrophysiological effects on rat hippocampal neurons, reduced spike frequency adaptation, possibly due to attenuation of certain K⁺ conductances. (Lampe, B. W. & Brown, B. S., 1991).

Studies in our laboratories have shown that for linopirdine and several structural analogs there is a good correlation between blockade of the M-current (a voltage dependent, receptor-sensitive outward potassium current) and enhancement of neurotransmitter release in vitro. Selective blockade of M-channels would result in pre-synaptic neurotransmitter release enhancement and augmentation of post-synaptic neurotransmitter effects, with minimal side effects resulting from activation of additional cellular mechanisms.

Although the pharmaceutical industry has targeted a variety of ion channels in development of therapeutic agents, the M-channel has received little attention. Located primarily in the brain, the physiological role of M-current is to suppress neuronal excitability. Blockade of M-current results in activation of neurotransmitter pathways. Agents which block M-channels would cause increases in neurotransmitter release and general brain excitation. These agents would, therefore, be useful in the treatment of neurological diseases involving either known neurotransmitter deficiencies (e.g., Alzheimer's disease, Parkinson's disease, depression, Huntington's disease), traumatic brain injury or the depressive phase of bipolar disorder.

The M-current is a slowly activating and deactivating potassium conductance that plays a critical role in determining the electrical excitability of neurons, controlling the subthreshold electrical excitability of neurons as well as the responsiveness to synaptic inputs (Brown 1998; Yamada et al., 1989; Wang and McKinnon, 1995). The M-current was first described in peripheral sympathetic neurons (“Brown and Adams, 1980; Constanti and Brown, 1981) and differential expression of this conductance produces subtypes of sympathetic neurons with distinct firing patters (Wang and McKinnon, 1995). The M-current is also expressed in many neurons in the central nervous system (Brown, 1988; Constanti, J., A. Sim, 1987; Storm, 1989; Womble and Moises, 1992).

To date, the molecular identity of the channels underlying the M-current remains unknown. The present invention demonstrates that KCNQ2 and KCNQ3 channel subunits can co-assemble to form a channel with essentially identical biophysical properties and pharmacological sensitivities as the native M-current and that the pattern of KCNQ2 and KCNQ3 gene expression is also consistent with these genes encoding the native M-current.

An object of present invention is to use KCNQ2 and KCNQ3 gene coexpression to screen for pharmacologically active compounds in high thoughput screening assays. The development of high throughput screening assays has tremendous commercial utility in the discovery of compounds useful in the treatment of neurological disorders.

A further object of the present invention is to provide a stable expression of the KCNQ2 and KCNQ3 genes in a mammalian cell line wherein either gene does not have to be retransfected into native cells each time an assay is to be performed. Stable expression in mammalian cells is advantageous over transient expression because transient expression is nonreplicating and the expression ceases when the cell expires.

### SUMMARY OF THE INVENTION

The present invention provides a method of testing a compound for utility in treating neurological disease wherein the compound demonstrates pharmacological activity as an agonist of the M-current or as an antagonist of the M-current. The method comprises contacting a compound with a mammalian cell that coexpresses a KCNQ2 gene and a KCNQ3 gene, wherein the KCNQ2 gene and the KCNQ3 gene form a voltage-gated potassium channel; and measuring the activity of the potassium channel.

Compounds which demonstrate pharmacological activity as an agonist of the M-current may be effective in the treatment of epilepsy, anxiety, insomnia or other hyperexcitability disorders. Compounds which demonstrate pharmacological activity as an antagonist of the M-current may be effective in the treatment of Alzheimer's disease, peripheral neuropathy or other neurodegenerative diseases.

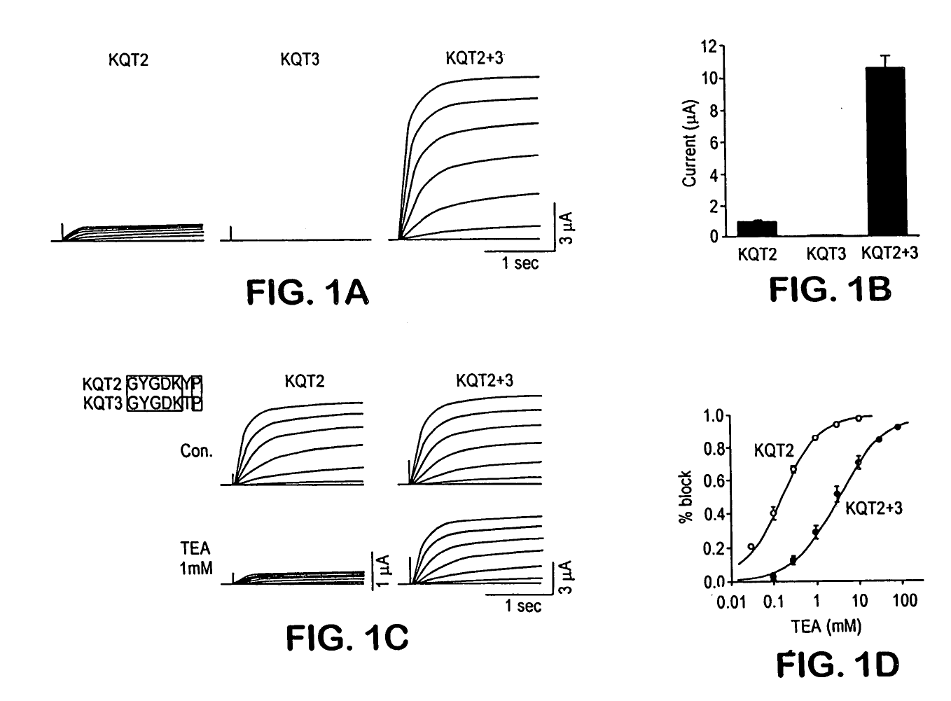

FIG. 1 illustrates that the KCNQ2 and KCNQ3 potassium channel subunit forms heteromultimers.

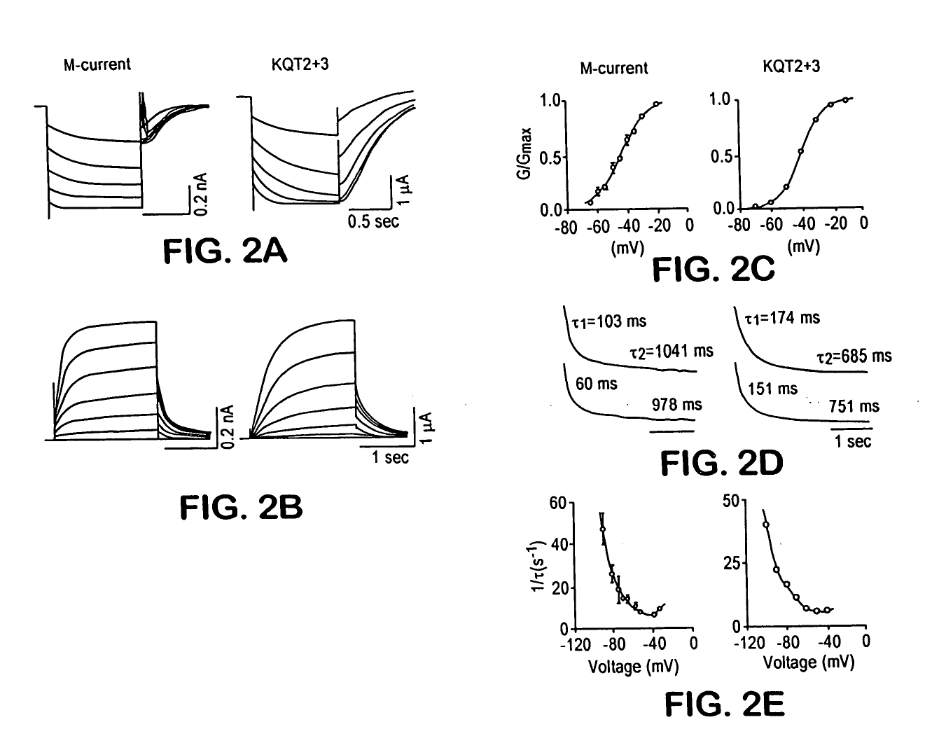

FIG. 2 illustrates the comparison of kinetic properties of native M-current in SCG neurons with KCNQ2+KCNQ3 heteromultimers.

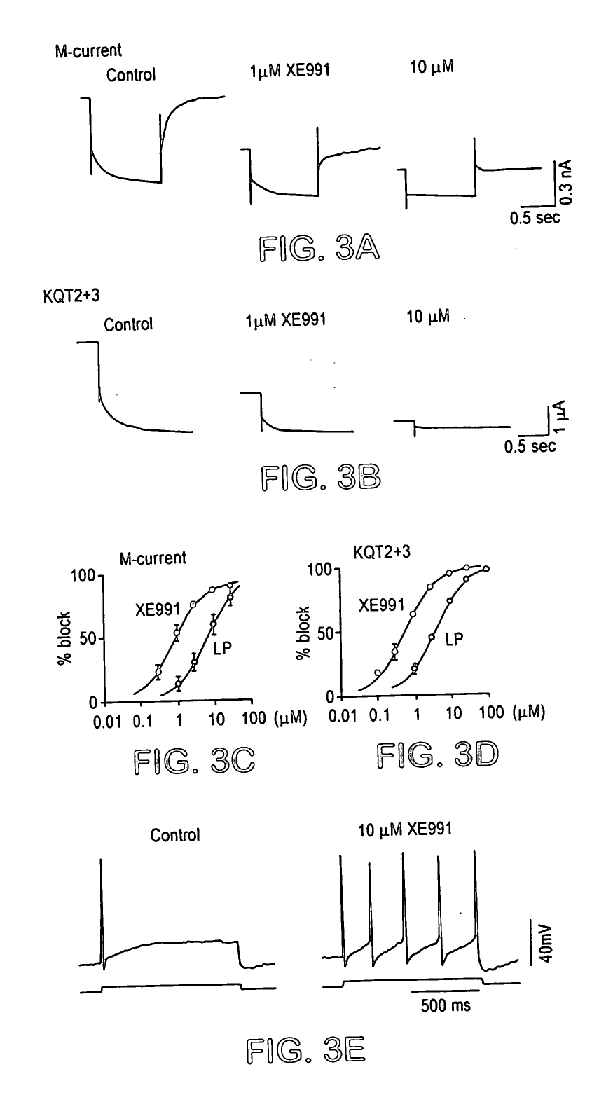

FIG. 3 illustrates that channel blockade by XE991 of the M-current and KCNQ2+KCNQ3 channels.

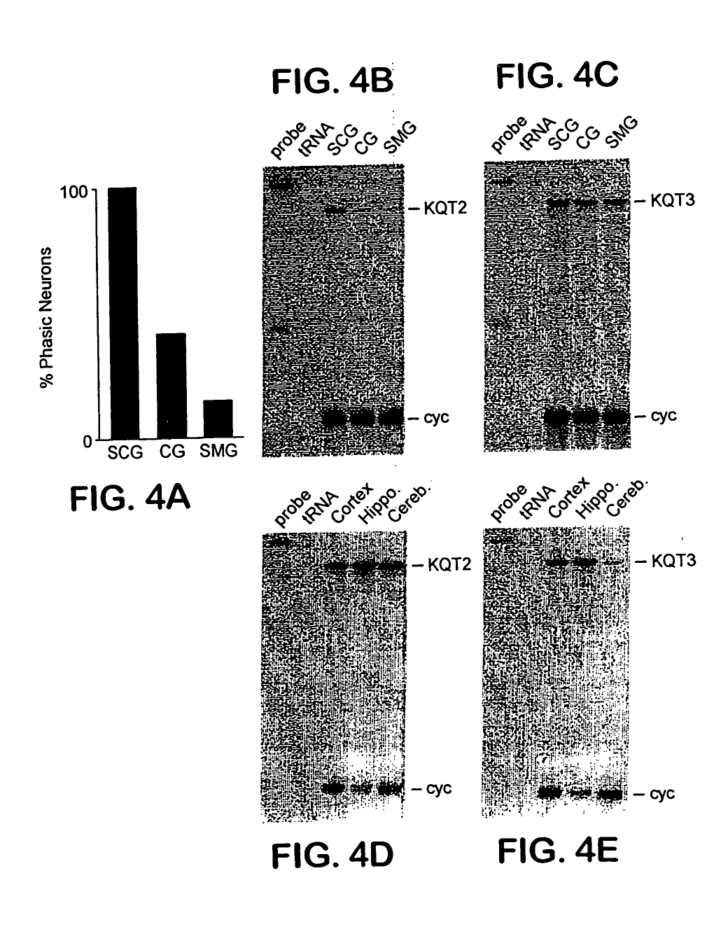

FIG. 4 illustrates KCNQ2 and KCNQ3 mRNA expression in different rat sympathetic ganglia and brain regions determined by RNase protection analysis.

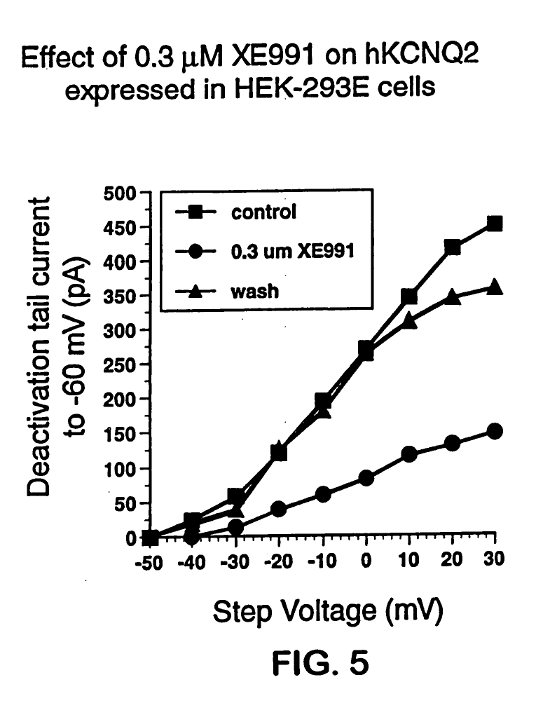

FIG. 5 illustrates the effect of 0.3 uM XE991 on hKCNQ2 expressed in a stable line of HEK-293E cells.

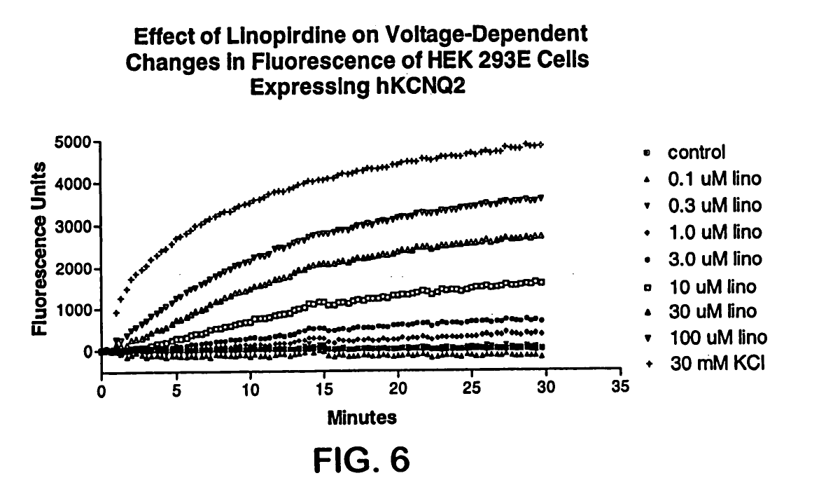

FIG. 6 illustrates that linopirdine induces a time- and concentration-dependent increase in flourescence of HEK 293E cells stably expressing the hKCNQ2 potassium channel.

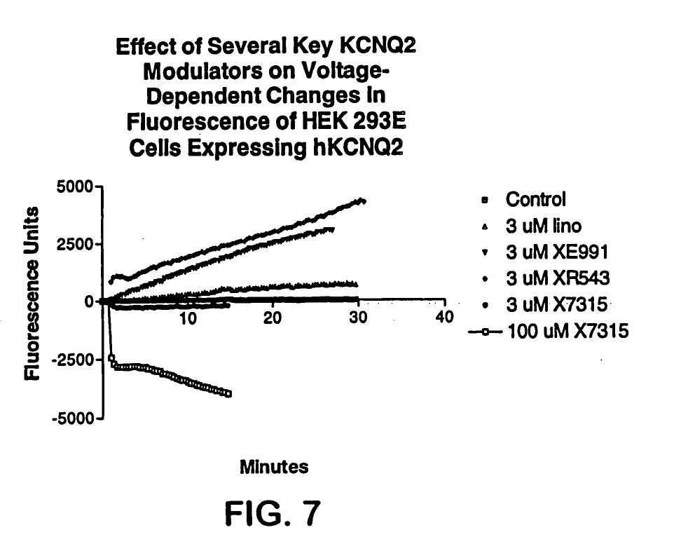

FIG. 7 illustrates the relative effects of several M-current modulators on the flourescence of HEK 293E cells stably expressing the hKCNQ2 potassium channel.

### DETAILED DESCRIPTION

In a first embodiment this invention is a method of evaluating a compound for utility in treating neurological disease comprising contacting a compound with a cell that coexpresses KCNQ2 and KCNQ3, wherein the KCNQ2 and the KCNQ3 form a potassium channel; and measuring the activity of the potassium channel.

It is preferred that the cell is an oocyte or a mammalian cell.

It is more preferred that the cell is a mammalian cell selected from HEK 293E, CHO and COS cells.

It is even more preferred that the cell is a mammalian HEK 293E cell.

It is also preferred that the KCNQ2 gene is hKCNQ2.

It is also preferred that the KCNQ3 gene is hKCNQ3.

It is also preferred that the compound is either an agonist of the potassium current or an antagonist of the potassium current.

It is also preferred that the activity of the potassium channel is measured by a current or a change in membrane voltage through a voltage sensitive dye wherein it is more preferred that the voltage sensitive dye is detectable by fluoresence.

In an even more preferred first embodiment this invention is a method of evaluating a compound for utility in treating neurological disease comprising contacting a compound with a mammalian cell that coexpresses hKCNQ2 and hKCNQ3, wherein the hKCNQ2 and the hKCNQ3 form a potassium channel; and measuring the current of the potassium channel.

It is even more preferred that the cell is a mammalian cell of the HEK 293E cell line.

It is also even more preferred that the compound is either an agonist of the potassium current or an antagonist of the potassium current.

In a second embodiment the present invention provides a pharmaceutical composition comprising a pharmaceutically acceptable carrier and a therapeutically effective amount of a compound identified by the screening assay of the invention or a pharmaceutically acceptable salt or prodrug form thereof, wherein said compound modulates a potassium channel formed by the coexpression of KCNQ2 and KCNQ3.

In a third embodiment the present invention provides a method for treating a degenerative neurological disorder involving a potassium channel formed by the coexpression of KCNQ2 and KCNQ3 comprising administering to a host in need of such treatment a therapeutically effective amount of a compound identified by the screening assay of the invention or a pharmaceutically acceptable salt or prodrug form thereof.

In a preferred embodiment the degenerative neurological disorder is epilepsy.

Compounds useful in the demonstration of the invention are linopirdine, XE991, and X7315. Linopirdine blocks the M-current in the micromolar concentration range by direct channel blockade (Aiken et al., 1995; Lamas et al., 1997; Costa and Brown, 1997). The IC₅₀ of Linopirdine is 7.0±1.1 μM. XE991 is 10,10-bis(4-pyridinylmethyl)-9(1OH)-anthracenone, has the formula:

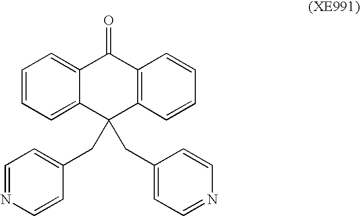

and is also a specific M-channel blocker. XE991 is well known in the art (see U.S. Pat. No. 5,173,489 example 440). The IC₅₀ of XE991 is 0.98±0.15 μM. X7315 which has the formula:

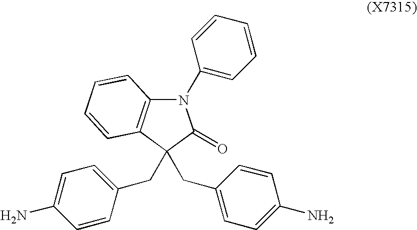

is a negative control and can be prepared by methods taught in the art and known to one skilled in the art of organic synthesis.

As used herein, a method of evaluating a compound for utility in treating neurological disease can be preformed in a number of ways. Any suitable container for contacting a compound to be evaluated with a cell in which the KCNQ2 and KCNQ3 gene has been expressed is envisaged. Such containers can be single or multiple. A preferred example is a well in a plate, preferrably a multiwell plate; especially a multiwell plate designed for high thoughput screening assays. The plate typically has 96 or 384 wells, but may have more, up to the limits of measuring activity in the well.

As used herein, a cell that coexpresses KCNQ2 and KCNQ3, is meant to mean any cell, mammalian or nonmammalian, wherein the KCNQ2 and the KCNQ3 can be expressed to form a voltage-gated potassium channel. All forms of KCNQ2 and KCNQ3 are envisaged which will correlate to the M-channel current. Equivalent forms of KCNQ2 and KCNQ3 may include nonmammalian, such as from Drosiphila or C. Elegans, or may be mammalian, such as forms from primate, rat or human. Human and rat are preferred, human is most preferred. It is preferred that the cell is an oocyte or a mammalian cell. It is more preferred that the mammalian cell be a HEK 293E, a CHO or a COS cell.

As used herein, measuring the activity of the potassium channel can be performed in a number of ways. One method of “measuring the activity” is to measure the current of the potassium channel, such methods are well known in the art. A second method envisaged for “measuring the activity” is through the use of a change in membrane voltage. Changes in membrane voltage can be determined by use of a voltage sensitive dye wherein it is preferred that the voltage sensitive dye is detectable by fluoresence. Example of a voltage sensitive dye is dibac. A third method for “measuring the activity” is ⁸⁶Rubidium efflux assay.

As used herein a compound which is an agonist of the potassium current is meant to be a compound which opens or increases the activity of the potassium channel expressed by the KCNQ2 and KCNQ3. As used herein a compound which is an antagonist of the potassium current is meant to be a compound which closes or blocks the activity of the potassium channel expressed by the KCNQ2 and KCNQ3. An example of an antagonist is linopirdine or XE991.

Injection of KCNQ2 mRNA results in the consistent expression of a slowly activating and deactivating potassium current (FIG. 1A) with properties similar to those described previously (Biervert et al., 1998). In contrast, the KCNQ3 gene does not encode a functional channel when expressed in oocytes by itself. When the KCNQ2 and KCNQ3 mRNAs are co-injected, however, the resultant current is 11-fold larger than that found in cells injected with KCNQ2 mRNA alone (FIG. 1B). The large increase in current density following co-injection of the KCNQ3 mRNA suggests that the KCNQ3 subunit facilitates expression of the KCNQ2 subunits, possibly by the formation of a heteromeric complex of KCNQ2 and KCNQ3.

In addition to affecting current density, the KCNQ3 subunit affects the sensitivity of the KCNQ2 channel to pharmacological blockade. The homomultimeric KCNQ2 channel was very sensitive to tetraethylammonium (TEA) (K_{D}=0.16±0.02 mM, n=5) whereas channels expressed following co-injection of KCNQ2 and KCNQ3 mRNAs were much less sensitive (K_{D}=3.5±0.7 mM, n=6). The KCNQ2 and KCNQ3 subunits differ within the pore region, at a position that determines sensitivity to blockade to TEA (FIG. 1C). The KCNQ2 subunit has a tyrosine residue at this position, which confers high sensitivity to TEA, whereas the KCNQ3 channel has a threonine residue, which confers low sensitivity to TEA (McKinnon and Yellen, 1990). The intermediate sensitivity to TEA block of the KCNQ2+KCNQ3 channels confirms that the KCNQ2 and KCNQ3 subunits co-assemble into a heteromultmeric complex (FIG. 1D), in a manner closely analogous to heteromultimers of Shaker channels (Heginbotham and McKinnon, 1992). For comparison, the native M-current in rat sympathetic neurons is also moderately sensitive to blockade by TEA (K_{D}=6.1 mM), as is the M-current found in hippocampal and olfactory cortex neurons (Constanti and Sim, 1987; Storm, 1989). It seems likely, therefore, that, if the KCNQ2 and KCNQ3 subunits contribute to the native M-channel, they assemble as a heteromultimeric complex with the expression of both subunits required to achieve both normal current levels and pharmacological properties.

The kinetic properties of the KCNQ2+KCNQ3 channel were remarkably similar to those of the native M-current. The M-current has the following characteristic kinetic properties: a relatively negative activation curve, a significant steady-state conductance at =30 mV and slow activation and deactivation kinetics (Wang and McKinnon, 1995; Constanti and Brown, 1981). Using the classic M-current protocol (Brown and Adams, 1980), the KCNQ2+KCNQ3 channel closely replicated the waveform of the native M-current (FIG. 2A). Deactivation and activation of the KCNQ2+KCNQ3 channel was slow and the channel was significantly activated at 30 mV. Activation was similar, both in terms of the shape of the current waveform and the rate of activation (FIG. 2B). The conductance-voltage curves were very similar for the two channel types with the threshold for activation near =60V and the majority of the channels activated at −30 mV (FIG. 2C). The deactivation kinetics of the M-current are biphasic (Marrion et al., 1992) and this was also true of the KCNQ2+KCNQ3 channel (FIG. 2D). Both channel types had similar time constants for the two components of deactivation. Deactivation time constants at −50 mV for the M-current were: 145±25 ms and 838±125 ms (55±3% fast component, n=4) and for KCNQ2+KCNQ3 were: 171±12 ms and 857±146 ms (49±3% fast component, n=9). At −60 mV, for the M-current, 126±28 ms and 934±117 ms (60±2% fast component, n=4), and for KCNQ2+KCNQ3 149±9 ms and 741±69 ms (59'3% fast component, n=9). The time constant of the fast component was voltage sensitive (FIG. 2E) whereas the slow component was relatively insensitive to voltage over the same voltage range.

While the kinetic properties of the KCNQ2+KCNQ3 channel were very similar to those of the native M-current it is important to establish other criteria that can be used to determine the molecular identity of the native conductance. The present invention demonstrates two compounds that are useful in establishing the identity of the M-channel: linopirdine and the compound 10,10-bis(4-pyridinylmethyl)-9(10H)-anthracenone (XE991). Linopirdine blocks the M-current in the micromolar concentration range by direct channel blockade (Aiken et al., 1995; Lamas et al., 1997; Costa and Brown, 1997). The IC₅₀ of XE991 is 0.98±0.15 μM (FIG. 3). Only one class of voltage-gated potassium channels had a similar pharmacological profile to that of the native M-current: the KQT channels, which were blocked by both XE991 and linopirdine at very similar concentrations to the native M-current (Table 1). Of particular interest is XE991, which has both high affinity and selectivity for both the native M-channel and KQT channels. No eag-related or Shaker-related channel tested had a similar sensitivity. Unlike the KCNQ2 and KCNQ3 channels, the KQT1 channel cannot contribute to the native M-channel because the KQT1 gene is not expressed in either the CNS (Wang et al., 1996) or sympathetic ganglia.

Consistent with the high selectivity of XE991 for the M-current is its effect on the firing properties of sympathetic neurons. In the rate, there are two classes of sympathetic neurons: phasic-firing neurons, which have a relatively large M-current, and tonic-firing neurons, which do not express and M-current (Wang and McKinnon, 1995). We have shown previously that differential expression of the M-current is the primary determinant of the different firing properties of phasic and tonic neurons (Wang and McKinnon, 1995). This conclusion is confirmed by the observation that blocking the M-current in phasic neurons with 10 μM XE991 converts the firing properties from phasic to tonic without otherwise affecting the electrophysiological properties of these cells (FIG. 3E).

The expression pattern of KCNQ2 and KCNQ3 genes in sympathetic ganglia is consistent with these genes encoding subunits of the M-channel. The expression of multi-subunit proteins is often regulated by limiting the expression of a single subunit and this is apparently true for the M-current. The superior cervical ganglia (SCG) contains only phasic neurons whereas the prevertebral sympathetic ganglia (celiac ganglia and superior mesenteric ganglia) contain predominantly tonic neurons (FIG. 4A). The gene regulating expression of the M-channel should, therefore, be expressed at significantly lower levels in the prevertebral sympathetic ganglia than in the SCG, and KCNQ2 gene expression does not in fact closely parallel M-current expression in these ganglia (FIG. 4B). Of the 24 different voltage-gated potassium channel genes tested to date, no other gene has a similar expression pattern in sympathetic ganglia (Dixon and McKinnon, 1996; Shi et al., 1997, Shi et al., 1998). The KCNQ3 gene was expressed at approximately equal levels in both SCG and prevertebral ganglia (FIG. 4C) and, since the KCNQ3 channel does not form a functional channel by itself, it is likely that M-current expression sympathetic ganglia is determined primarily by regulation of KCNQ2 gene expression.

In the CNS, the M-current is expressed in many neurons in the cortex and hippocampus but is relatively rare in the cerebellum. In contrast to the peripheral nervous system, KCNQ2 gene expression does not parallel M-current expression in these CNS regions and the KCNQ2 gene was expressed at relatively high levels in all three regions (FIG. 4D). The KCNQ3 gene, however, was expressed at much lower levels in the cerebellum than in cortex and hippocampus, like the M-current (FIG. 4E), suggesting that regulation of KCNQ3 gene expression is also important in determining M-current expression levels in vivo. This conclusion is consistent with the in vitro results demonstrating that the KCNQ2+KCNQ3 heteromultimeric channel expresses much more efficiently than does the KCNQ2 homomultimer.

Taken together, these results strongly suggest that the KCNQ2 and KCNQ3 subunits contribute to the native M-channel. The KCNQ2+KCNQ3 channel is the only known potassium channel that can reproduce the unique kinetic properties of the native M-current and several different pharmacological agents have very similar effects on the native M-current and the KCNQ2+KCNQ3 channel. In particular, the compound XE991 is highly selective for both the M-current and the KQT channels. Finally, the KCNQ2 gene is the only known potassium channel gene that is expressed in a pattern that parallels the distribution of the M-current in peripheral sympathetic ganglia. These data make a compelling case of the hypothesis that the KCNQ2+KCNQ3 channel is a molecular correlate of the M-current in sympathetic neurons.

The KCNQ2 and KCNQ3 genes are also abundantly expressed in the CNS and it is likely that the KCNQ2+KCNQ3 subunits contribute to the M-current in central neurons. This conclusion is consistent with the observation that mutations in either the KCNQ2 or KCNQ3 genes result in an inherited autosomal dominant epilepsy (Biervert et al., 1998; Singh et al., 1998; Charlier et al., 1998). The very similar phenotypes produced by mutations in either of these two distinct genes can be explained by the observation that both gene products are required to product full expression of functional channels. Identification of the physiological function of the channel encoded by the KCNQ2 and KCNQ3 genes may facilitate the development of symptomatic treatments for these epilepsies.

### EXAMPLE 1

FIG. 1. The KCNQ2 and KCNQ3 potassium channel subunit form heteromultimers.

A. Currents recorded in Xenopus oocytes following injection of KCNQ2 mRNA, KCNQ3 mRNA or an equimolar ratio of KCNQ2 and KCNQ3 mRNAs. Currents elicited by 2 second voltage steps from a holding potential of −70 mV over the range of −60 to 0 mV in 10 mV increments. The currents in KCNQ3 mRNA injected oocytes were not significantly larger than those seen in un-injected cells.

Method: Full-length KCNQ2 cDNAs were amplified from adult human brain cDNA using the following primers (CCCCGCTGAGCCTGAG, TGTAAAAGGTCACTGCCAGG) with the Expand Fidelity enzyme mixture (Boehringer Mannheim, Indianapolis). The KCNQ2 cDNA clone used in the biophysical studies was identical to the KCNQ2 cDNA isolated previously from a fetal brain cDNA library (Singh et al., 1998) except that it had a small deletion in the carboxy intracellular domain (30 amino acids from residues 417 to 446). This region is also alternatively spliced in the KCNQ2 cDNA clone described by Biervert et al. (1998). Preparation, injection of cRNA and recording from oocytes was performed at room temperature as described previously (Dixon et al. 1996). The standard extracellular recording solution contained: 82 mM NaCl, 2 mM KCl, 1.8 mM CaCl₂, 1 mM MgCl₂, and 5 mM Na-HEPES (pH 7.6). Data collection and analysis were performed using pClamp software (Axon Instruments, Foster City, Calif.).

B. Histogram showing the average current response to a voltage-clamp step to 0 mV from −70 mV in cells injected with KCNQ2, KCNQ3 or an equimolar ratio of KCNQ2 and KCNQ3 mRNAs (45 ng of each mRNA was injected per oocyte). Average current responses in the three sets of cells were significantly different to each other (p<0.001, n=19−22).

C. Effect of 1 mM TEA on currents elicited from oocytes injected with KCNQ2 mRNA or an equimolar ratio of KCNQ2 and KCNQ3 mRNAs. Same voltage clamp protocol to that used in (A). The KCNQ2+KCNQ3 mRNA mixture was diluted to reduce current density. Inset shows a comparison of the deduced amino acid sequence in the pore region around the residue controlling TEA sensitivity (equivalent to position 449 in the Shaker H4 channel (MacKinnon and Yellen, 1990).

D. Dose response curves for TEA block of KCNQ2 channels and KCNQ2+KCNQ3 channels. Figure shows averaged data fitted with the Hill equation using average parameters obtained from fits to individual cells. For KCNQ2: K_{D}=0.16±0.02 mM (n=5) and the Hill coefficient was set to unity. For KCNQ2+KCNQ3: KD=3.5±0.7 mM, Hill coefficient=0.82±0.03 (n=6).

### EXAMPLE 2

FIG. 2. Comparison of kinetic properties of native M-current in SCG neurons with KCNQ2+KCNQ3 heteromultimers.

A. Current response to traditional M-current voltage clamp protocol for native current and KCNQ2+KCNQ3 channels. Holding potential was −30 mV and membrane potential was stepped to more negative potentials for 1 second in 10 mV increments. Apparent differences in the current waveforms are largely due to the presence of a significant linear leak current in the recordings from SCG neurons that is relatively smaller in the oocytes. The initial phase of M-current reactivation in SCG neurons is obscured by activation of the A-current.

Method: Recordings of the M-current in sympathetic neurons in intact ganglia were performed at room temperature as described previously (Wang and McKinnon, 1995). The standard extracellular recording solution was: NaCl (133 mM, KCl (4.7 mM), NaH₂PO₄ (1.3 mM), NaHCO₃ (16.3 mM), CaCl₂ (2 mM), MgCl₂ (1.2 mM) and glucose (1.4 g/liter), bubbled with 95% O₂-5% CO₂ to give pH 7.2-7.4.

B. Activation of M-current and KCNQ2+KCNQ3 channels from a holding potential of −60 mV in 5 mV increments.

C. Conductance-voltage curves fitted with a single Boltzmann function. For the native M-current the fit is to averaged data points, with Vₙ=−44 mV and kₙ=−8.8 mV (n=6, bars are s.e.m.). Conductance-voltage curves for KCNQ2+KCNQ3 channels, Vₙ=−40 +1 mV and kₙ=−6.8±0.1 mV (n=6, bars are s.e.m.). Conductance-voltage curves for KCNQ2+KCNQ3 channels were constructed using tail currents at −60 mV.

D. Deactivation process had two time constants for both channel types. Time constants for deactivation are shown next to current traces for steps from −30 mV holding potential to −50 mV or −60 mV. Biexponential fits are superimposed on the experimental data.

E. Reciprocal time constant for fast deactivation of the native M-current and KCNQ2+KCNQ3 channels. Data points are averages from 3 to 9 cells for the native M-current and 9 cells for KCNQ2+KCNQ3. Data were fitted with the equation (Constanti and Brown, 1981): 1/τ=∝₀(β₀)(exp(±(V_{m=V}₀)/y)), where Vₘ is the membrane potential, ∝₀(β₀)=3.8 sec⁻¹, V₀=−45.4 mV and y=18.3 mV for the native M-current and ∝₀(β₀)=3.0 sec⁻¹, V₀=−46.7 mV and y=20.9 mV for the KCNQ2+KCNQ3 channel. The native M-current was recorded from SCG neurons in intact, isolated ganglia and the KCNQ2+KCNQ3 currents were recorded in Xenopus oocytes, both at room temperature.

### EXAMPLE 3

FIG. 3. Channel blockade by XE991 of the M-current and KCNQ2+KCNQ3 channels.

A. Blockade of M-current in SCG neurons by XE991. Holding potential was −30 mV and step potential was −50 mV for 1 second. The shift in holding current following drug application is due to the inhibition of M-current activated at the holding potential.

B. Blockade of KCNQ2+KCNQ3 channels by XE991. Holding potential was −60 mV and the cell was repetitively depolarized to −30 mV for 1 minute to reach steady-state blockade. Tail currents were recorded at −50 mV.

C and D. Dose-response curves for linopirdine (open symbols) and XE991 (closed symbols) for blockade of M-current (C) and KCNQ2+KCNQ3 channels (D). Maximal block of native M-current was 93±2%. Data points are averages and error bars represent s.e.m.

E. Effect of 10 μM XE991 on the firing properties of phasic sympathetic neuron recorded from the SCG. Membrane potential was held at −60 mV and the depolarizing current step was 0.2 nA for control and XE991 application.

### EXAMPLE 4

FIG. 4. KCNQ2 and KCNQ3 mRNA expression in different rat sympathetic ganglia and brain regions determined by RNase protection analysis.

A. Histogram showing the distribution of phasic neurons in prevertebral and paravertebral sympathetic ganglia. Neurons in SCG are exclusively phasic (n=36) whereas only 42% of the neurons in the CG and 15% in SMG are phasic (n=52 and 40 respectively). (Data adapted from Wang and McKinnon (1995)).

B. KCNQ2 mRNA expression in sympathetic ganglia. Samples tested were prepared from superior cervical ganglia (SCG), celiac ganglia (CG) and superior mesenteric ganglia (SMG). KCNQ2 expression in the CG and SMG was 30% and 19% respectively relative to expression in the SCG (average of two experiments).

Method: Preparation of RNA, RNase protection assays and isolation of specific rat KCNQ2 and KCNQ3 probes were performed as described previously (Dixon and McKinnon, 1996). RNA expression was quantitated directed from dried gels with a Phosphorlmager (Molecular Dynamics, Sunnyvale, Calif.).

C. KCNQ3 mRNA expression in sympathetic ganglia.

D. KCNQ2 mRNA expression in three brain regions. Samples tested were prepared from cortex, hippocampus (Hippo.) and cerbellum (Cereb.).

E. KCNQ3 mRNA expression in three brain regions. All samples contained μg total RNA, the cyclophilin gene (cyc) was used as a positive internal control and yeast tRNA as a negative control.

### EXAMPLE 5

Using the method of the invention, the selective blocking of the M-channel is demonstrated in oocytes. Table 1 demonstrates the pharmacological profile for linopirdine and the XE991 as selective blockers. XE991 demonstrates both high affinity and selectivity for both the native M-channel and KQT channels.

IC₅₀ values are all expressed in μM, mean±s.e.m. In cases where the IC₅₀ values were greater than 100 μM the exact value is not reported due to limited solubility in the drug. It has been suggested that eag-related potassium channels might encode the M-current (Stansfeld et al., 1997) and all the eag-related channels expressed in SCG (Shi et al., 1997; 1998), were tested in addition to representative examples of delayed rectifier and A-channels.

TABLE 1
Comparison of M-Current and Cloned Potassium Channels:
IC₅₀ for Linopirdine and XE991 Blockade

| | XE991 | | Linopirdine | |
|---|---|---|---|---|
| M-current | 0.98 ± 0.15 | (n = 3) | 7.0 ± 1.1² | (n = 5) |
| KCNQ2 + KCNQ3 | 0.6 ± 0.1 | (n = 6) | 4.0 ± 0.5 | (n = 6) |
| KCNQ2 | 0.71 ± 0.07 | (n = 6) | 4.8 ± 0.6 | (n = 5) |
| KQT1 | 0.75 ± 0.05 | (n = 7) | 8.9 ± 0.9 | (n = 6) |
| eag1 | 49 ± 6¹ | (n = 6) | 31 ± 3¹ | (n = 9) |
| erg1 | >100 | (n = 4) | 53 ± 4 | (n = 6) |
| erg3 | >100 | (n = 6) | 85 ± 5 | (n = 5) |
| elk1 | >100 | (n = 5) | 37 ± 4³ | (n = 7) |
| Kv1.2 | >100 | (n = 5) | 68 ± 6 | (n = 4) |
| Kv4.3 | 43 ± 7 | (n = 5) | 86 ± 14 | (n = 4) |

¹Blockade of the eag1 channel was incomplete with 82 ± 1% (n = 4) blockade by 1 mM linopirdine and 56 ± 2% (n = 6) blockade by 100 μM XE991.
²Data adapted from Costa and Brown, a similar value of 3.4 ± 0.3 μM was obtained by Lamas et al . . .
³The IC₅₀ for block of elk1 channels by linopirdine was very voltage dependent and the value shown is for a step to −10 mV. IC₅₀ values ranged from 26 ± 3 μM at −20 mV (n = 7) to 144 ± 10 μM at +30 mV (n = 3).

### EXAMPLE 6

Isolation of hKCNO2 and rKCNO3 cDNA

Full-length hKCNQ2 cDNAs were amplified from adult human brain cDNA using standard molecular biology techniques and the following primers (CCCCGCTGAGCCTGAG, TGTAAAAGGTCACTGCCAGG) with the Expand Fidelity enzyme mixture (Boehringer Mannheim, Indianapolis, Ind.). The cDNA clone used in biophysical and pharmacological studies was identical to the hKCNQ2 cDNA previously isolated from a fetal brain cDNA library by Singh, et al. (1998) except for a small deletion in the carboxy-terminal intracellular domain resulting in a 30 amino acid deletion of residues 417-446.

PCR amplification of partial rKCNQ3 CDNA clones from rat brain and rat superior cervical ganglia (SCG) CDNA was performed. An initial sequence encompassing the entire open reading frame of the rKCNQ3 gene was determined through several rounds of 5′ and 3′ RACE PCR using initial anchor oligonucleotides complementary to the partial cDNA clone and SCG cDNA as a template for amplification. Once cDNAs were obtained that extended beyond both the 5′ and 3′ ends of the open reading frame, oligonucleotides complementary to non-coding regions at either end of the coding sequence were designed. Multiple full-length cDNA clones were amplified in independent PCR reactions from rat SCG cDNA using Expand Long Template PCR (Boehringer Mannheim, Indianapolis, Ind.) using several combinations of the following oligonucleotides: forward (TTGACTCCCCATCCGACCT; GCCTTTGCCTTCTTTTGGG), reverse (ACCGCGCACATGCATG, GTGACATGGGGAGGAAGAA). Four independent clones were sequenced in their entirety in both directions by automatic sequencing (GenBank accession number AF091247).

### EXAMPLE 7

Expression of hKCNO2 and hKCNO3 CDNA

HEK 293E Cells

The human KCNQ2 cDNA was subcloned between the StuI and XbaI restriction sites of expression vector phchm3 AR (Shen et al., 1995), creating the hKCNQ2 expression vector pm3 AR-hKCNQ2-1. This vector is a modification of pHEBo, an Epstein Barr Viral origin of replication plasmid (Sudgen et al., 1985) into which a CMV immediate early promoter, a multicloning site, and the SV40 small t intron and early poly adenylation signal regions have been added. This plasmid replicates episomally in primate cells expressing the EBV nuclear antigen 1 (Shen et al., 1995).

A stable cell line expressing hKCNQ2 was established by transfecting the plasmid pm3AR-hKCNQ2-1 into 293 EBNA cells (Invitrogen) using Effectene (Qiagen). This plasmid contains the hKCNQ2 gene under the control of the CMV immediate early promoter, the EBV oriP for maintenance of the plasmid as an extrachromosomal element in the appropriate cells (nonrodent mammalian cells expressing EBNA), and the hph gene from E.coli to yield resistance to hygromycin B. 293 EBNA cells were grown in Dulbecco's modified Eagle medium containing 10% fetal bovine serum at 37° C. in a humid environment with 5% C0₂. Cells were plated at a density of 1-3×10⁵ cells/well in a 6-well plate and transfected with 0.4 ug of plasmid per well the following day. Within 24 hrs after transfection, the cells were expanded into T75 flasks; within 24 hours after expansion, hygromycin B was added to the media at a concentration of 250 ug/ml to select for transfectants. All cells resistant to hygromycin B after a week must be harboring the plasmid. No chromosomal integration is necessary for the maintenance of this plasmid. All transfected cells are essentially the same, eliminating the need for subcloning.

It is understood that one skilled in the art, using the methods taught herein and methods known in the literature, could construct a stable mammalian cell line that coexpresses KCNQ2 and KCNQ3 channels, preferably hKCNQ2 and hKCNQ3 channels.

### EXAMPLE 8

Part A. Assay: Determination of Functional Expression

To determine whether the hygromycin B resistant HEK 293E cells express functional hKCNQ2 channels, the presence of potassium currents was evaluated using the perforated-patch voltage-clamp technique. Briefly, cells growing on plastic Petri dishes or poly-D-lysine-coated glass coverslips in DMEM (with 10% FBS and 250 ug/ml hygromycin B) at 37° C. and 5% CO₂ were removed from the incubator and allowed to reach room temperature. Growth medium was replaced with a bathing solution containing, in mM: 140 NaCl, 3 KC1, 2.5 CaCl₂, 1 MgCl₂, 10 HEPES, 10 dextrose, pH adjusted to 7.2-7.4 with NaOH and osmolarity adjusted to 305-310 mosm with water. Voltage-clamp recordings were obtained using 2-5 Mohm resistance microelectrodes that were pulled from borosilicate glass (1.5 mm OD/1.0 mm ID; World Precision Instruments, Sarasota, Fla.) on a Sutter P-80/PC electrode puller (Sutter Instruments, Novato, Calif.) and filled with intracellular solution containing, in mM: 130 Kaspartate, 10 KCl, 1 CaCl₂, 2 MgCl₂, 10 HEPES, 10 K₄BAPTA, 5 K₂ATP and pH adjusted to 7.3 with 1 N NaOH. Freshly prepared amphotericin B was added to the intracellular solution (at a final concentration of 250 ug/ml) on the day of each experiment. After waiting for electrical access to the cell to be established (typically 5-15 minutes), current recordings were obtained by means of an Axopatch 1C or Axopatch 200A amplifier (Axon Instruments, Foster City, Calif.) using pClamp software (version 6.0.3, Axon Instruments) and a Pentium II IBM-compatible computer. From a holding potential of −60 mV, the cell is voltage clamped in 5-10 mV increments to 0-30 mV for 1-3 sec and subsequently returned to holding potential. The presence of a rapidly activating, non-inactivating outward current during the depolarizing step and a mono- or bi-phasic deactivation tail current upon repolarization to holding potential indicates the functional expression of hKCNQ2. Alternatively, a holding potential of −30 mV to 0 mV can be used with the presence of a mono- or bi-phasic deactivation tail current upon imposition of a 20-30 mV hyperpolarizing pulse will also indicate the functional expression of hKCNQ2. Using the same recording procedures on cells coexpressing KCNQ2 and KCNQ3, current amplitude is likely to be markedly increased, as was observed in oocytes (Wang et al., 1998).

Part B. Assay: Determination of Compound Activity

Electrophysiological Determination

Stock solution(s) of test compound(s) was (were) prepared immediately before use. An aliquot of the stock solution was diluted with an appropriate volume of bathing solution (as defined above) to attain a final working concentration(s) of test compound(s). Generally, the stock solution is about 0.1 mM to about 100 mM in DMSO and the aliquot is about 5 to about 100 uL, but it is understood that the concentration and volumes are not limited to these ranges for one skilled in the art can readily determine the appropriate concentrations and volumes dependening on the activity of the compound in the assay and the sensitivity of detection. Recording of current through KCNQ2+KCNQ3 channels as described above is performed prior to and at various times during the perfusion of the cell with a test compound solution. Current recordings can subsequently be made while perfusing the cell with a drug-free solution to determine reversibility of drug effects. Comparison of current amplitudes before test compound administration with those during administration indicate whether an agent affects channel activity. An example of an agent, XE991, that reversibly blocks hKCNQ2 expressed in HEK 293E cells is illustrated in FIG. 5.

A compound is considered active in the assay if the current changes more than 15%.

Determination of Membrane Potential-evoked Fluorescence

The ability of KCNQ2 modulators to alter membrane potential was assessed using a fluorescence imaging plate reader (FLIPR). Cultured HEK 293E cells expressing hKCNQ2 channels are plated on poly-D-lysine-coated 96-well Costar microplates (#3603) at 300,000-750,000 cells/ml and allowed to reach confluence in DMEM (with 10% FBS and 250 ug/ml hygromycin B) at 37° C. and 5% CO₂. After reaching confluence, growth medium is removed, replaced with a standard Hank's balanced salt solution containing 5 uM dibac₍₄₎3, bis-oxonol (Molecular Probes # B-438) at 37° C. The cell plate is then placed in the recording chamber (equilibrated to 37° C.) of a fluorescence imaging plate reader (FLIPR; Molecular Devices) along with two drug plates (one containing standard Hank's balanced salt solution+dibac and the other containing standard Hank's balanced salt solution+dibac+test compound at various experimental concentrations). The FLIPR is programmed to take baseline fluorescence readings to ensure thermal stability, add the standard Hank's balanced salt solution+dibac solution to the cell plate and take fluorescence readings to demonstrate membrane depolarization (required for opening of KCNQ2 channels), add standard Hank's balanced salt solution+dibac+test compound and take fluorescence readings to determine whether the test compound blocks the channel (indicated by an increase in fluorescence) or opens the channel (indicated by a decrease in fluorescence).

### EXAMPLE 9

Using the method of the invention, the selective blocking of the M-channel is demonstrated in HEK 293E cells stably expressing the hKCNQ2 potassium channel. FIG. 6 illustrates that linopirdine induced a time- and concentration-dependent increase in flourescence of HEK 293E cells stably expressing the hKCNQ2 potassium channel. These cells were loaded with the voltage-sensitive fluorescent dye, DiBAC, that distributes across cell membranes in a voltage-dependent manner. As cells depolarize (become more positive inside), more dye enters the cells and an increase in fluorescence occurs. Thus, these results indicate that under the conditions of the assay, linopirdine induced a time- and concentration-dependent depolarization which is believed to be mediated through a blockade of hKCNQ2.

### EXAMPLE 10

Using the method of the invention, the selective blocking of the M-channel is demonstrated in HEK 293E cells stably expressing the hKCNQ2 potassium channel. In FIG. 7, the relative effects of several M-current modulators on the flourescence of HEK 293E cells stably expressing the hKCNQ2 potassium channel is shown. These cells were loaded with the voltage-sensitive fluorescent dye, DiBAC, that distributes across cell membranes in a voltage-dependent manner. As cells depolarize, more dye enters the cells and an increase in fluorescence occurs. Conversely, as cells hyperpolarize (become more negative inside), more dye leaves the cells and a decrease in fluorescence occurs. Thus, these results indicate that, at 3 μM, XE991 and XR543 induced more membrane depolarization than linopirdine. In addition, at 3 μM, X7315 exerted no effect whereas, at 100 μM, X7315 induced an apparent membrane hyperpolarization. These results suggest that this fluorescence assay can be utilized to detect both blockers and openers of the hKCNQ2 channel.

### UTILITY

The two genes, hKCNQ2 and hKCNQ3, synergisticly or separately, are believed to have a possible role in neurological disease. Mutations in the pore, sixth membrane-spanning domain or C-terminal regions of hKCNQ2 and in the pore region of hKCNQ3 have been identified in BFNC (benign familial neonatal convulsions) patients (Biervert, et al., 1998; Singh, et al., 1998; Charlier et al., 1998). In hKCNQ2, a five base pair insertion, which causes the deletion of >300 amino acids from the intracellular carboxy terminus, results in the expression of a channel which lacks the wild-type capability of conducting outward potassium current (Biervert, et al., 1998). These findings suggest that mutations causing a functional deficit in hKCNQ2 or hKCNQ3 may play a causative role in some forms of human epilepsy. It is possible, therefore, that pharmacological agents which re-establish functionality to mutated forms of hKCNQ2 and hKCNQ3 may be an effective treatment for BFNC. Given the apparent role of these channels serving as the molecular correlate of the M-current (Wang et al., in press), long known to play an important role in regulating neuronal excitability (Brown, 1988), agents which enhance the function of the wild-type channel may represent novel treatments for the more commonly observed partial and tonic-clonic seizures, in addition to other hyperexcitability disorders such as anxiety and insomnia. Similarly, antagonists of these channels may be effective in the treatment of Alzheimer's disease, peripheral neuropathy or other neurodegenerative diseases. A potential role for the KCNQ2 channel in regulating neuromuscular function is supported by the recent report of its presence in motor neurons of the spinal cord (Dworetzky, et al., 1998).

### Dosage and Formulation

The compounds determined from the present invention can be administered using any pharmaceutically acceptable dosage form known in the art for such administration. The active ingredient can be supplied in solid dosage forms such as dry powders, granules, tablets or capsules, or in liquid dosage forms, such as syrups or aqueous suspensions. The active ingredient can be administered alone, but is generally administered with a pharmaceutical carrier. A valuable treatise with respect to pharmaceutical dosage forms is Remington's Pharmaceutical Sciences, Mack Publishing.

The compounds determined from the present invention can be administered in such oral dosage forms as tablets, capsules (each of which includes sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed to prevent or treat neurological disorders related to modulation of a potassium channel, more specifically the M-current, formed by expression of KCNQ2 and KCNQ3 genes, such as epilepsy, anxiety, insomnia, or Alzheimer's disease.

The compounds of this invention can be administered by any means that produces contact of the active agent with the agent's site of action in the body of a host, such as a human or a mammal. They can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice.

The dosage regimen for the compounds determined from the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient,and the effect desired. An ordinarily skilled physician or veterinarian can readily determine and prescribe the effective amount of the drug required to prevent, counter, or arrest the progress of the condition.

Advantageously, compounds determined from the present invention may be administered in a single daily dose, or the total daily dosage may be administered in divided doses of two, three, or four times daily.

The compounds identified using the present invention can be administered in intranasal form via topical use of suitable intranasal vehicles, or via transdermal routes, using those forms of transdermal skin patches wall known to those of ordinary skill in that art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or β-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds determined from the present invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds determined from the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like.

The pharmaceutically acceptable salts of the present invention can be synthesized from the parent compound identified from the screening assay which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Bibliography

Lampe, B. W. & Brown, B. S. (1991). Electrophysiological effects of DuP 996 on hippocampal CA1 neurons. Soc. Neurosci. Abstr., 17, 1588.

Shen, E. S., Cooke, G. M., & Horlick, R. A. (1995). Improved expression cloning using receptor genes and Epstein-Barr virus ori-containing vectors. Gene 156: 235-239.

Sudgen, B., Marsh, K., & Yates, J. L. (1985). A vector that replicates as a plasmid and can be efficiently selected in B-lymphoblasts transformed by Epstein-Barr virus. Mol. Cell. Biol. 5: 410-413.

Yang, W-P., Levesque, P. C., Little, W. A., Conder, M. L., Ramakrishnan, P., Neubauer, M. G. & Blaner, M. A. (1998). Functional expression of two KvLQTI-related potassium channels responsible for an inherited idiopathic epilepsy. J. Biol. Chem. 273: 1941919423.

Charlier, C., Singh, N. A., Ryan, S. G., Lewis, T. B., Reus, B. E., Leach, R. J. & Leppert, M. (1998). A pore mutation in a novel KQT-like potassium channel gene in an idiopathic epilepsy family. Nature Genetics 18: 53-55.

Biervert, C., Schroeder, B. C., Kubisch, C., Berkovic S. F., Propping, P., Jentsch, T. J. & Steinlein, O. K. (1998). A potassium channel mutation in neonatal human epilepsy. Science 279: 403-406.

Singh, N. A., Charlier, C., Stauffer, D., DuPont, B. R., Leach, R. J., Melis, R., Ronen, G. M., Bjerre, I., Quattlebaum, T., Murphy, J. V., McHarg, M. L., Gagnon, D., Rosales, T. O., Peiffer, A., Anderson, V. E. & Leppert, M. (1998). A novel potassium channel gene, KCNQ2, is mutated in an inherited epilepsy of newborns. Nature Genetics 18: 2529.

Dworetzky, S. I., Trojnacki, J. T., Goldstein, M., Boissard, C., Weaver, C. D., Rose, G. & Gribkoff, V. K. (1998). Cloning and expression of mouse KCNQ2: A nervous-system specific voltage-gated potassium channel. Soc. Neurosci. Abstracts 24: 2023.

Brown, D. A. (1988) M-Currents: An update. Trends Neurosci. 11: 294-299.

Wang, H-S., Pan, Z., Sh@ W., Brown, B. S., Wymore, R. S., Cohen, I. S., Dixon, J. E. & McKinnon, D. (in press). The KQT2 and KQT3 potassium channel subunits: Molecular correlates of the M-channel. Science (accepted for publication).

D. A. Brown, in Ion Channels. T. Narahashi, Ed. (Plenum, New York, 1988), pp. 55-94.

W. M. Yamada, C. Koch, P. R. Adams, in Methods in Neuronal Modeling, C. Koch and I. Segev, Eds. (Bradford, Cambridge, 1989), pp. 97-133.

H. S. Wang, D. McKinnon, J. Physiol. 485, 319 (1995).

D. A. Brown, P. R. Adams, Nature 283, 673 (1980)

A. Constanti, D. A. Brown, Neurosci Lett. 24, 289 (1981)

J. F. Storm, J. Physiol, 409, 171 (1989); A. Constanti, J. A. Sim, J. Physiol. 387, 173 (1987).

M. D. Womble, H. C. Moises, J. Physiol. 457, 93 (1992)

Q. Wang et al., Nature Genetics 12, 17 (1996).

A. Wei, T. Jegla, L. Salkoff, Neuropharmacol. 35, 805 (1996).

N. A. Singh et al., Nature Genetics 18, 25 (1998).

C. Biervert et al., Science 279, 403 (1998).

C. Charlier et al., Nature Genetics 18, 53 (1998).

M. C. Sanguinetti et al., Nature 384, 80 (1996).

J. Barhanin et al., Nature 384, 78 (1996).

R. MacKinnon, G. Yellon, Science 250, 250 (1990).

L. Heginbotham, R. MacKinnon, Neuron 8, 483 (1992).

N. V. Marrion, P. R. Adams, W. Gruner, Proc. R. Soc. Lond. B 248, 207 (1992).

J. F. Cassell, E. M. McLachlan, Br. J. Pharmacol. 91, 259 (1987); H. S. Wang, D. McKinnon, J. Physiol. 492, 467 (1996).

S. P. Aiken, B. J. Lampe, P. A. Murphy, B. S. Brown, Br. J. Pharmacol. 115, 1163 (1995).

J. A. Lamas, A. A. Selyanko, D. A. Brown, Eur. J. Neurosci. 9, 605 (1997).

A. M. N. Costa, B. S. Brown, Neuropharmacol. 36, 1747 (1997).

J. E. Dixon et al., Circ. Res. 79, 659 (1996).

J. E. Dixon, D. McKinnon, Eur. J. Neurosci. 8, 183 (1996).

C. E. Stansfeld et al., Trends Neurosci. 20, 13-14 (1997).

W. Shi et al., J. Neurosci. 17, 9423 (1997); W. Shi et al., J. Physiol., 511, 675 (1998).

What is claimed is:

1. A method of evaluating a compound for utility in treating neurological disease comprising contacting a compound with a cell that coexpresses KCNQ2 and KCNQ3, wherein the KCNQ2 and the KCNQ3 form a potassium channel; and measuring the activity of the potassium channel.

2. The method of claim 1 wherein the cell is an oocyte.

3. The method of claim 1 wherein the cell is a mammalian cell.

4. The method of claim 1 wherein the cell is a mammalian cell selected from HEK 293E, CHO and COS cells.

5. The method of claim 1 wherein KCNQ2 is hKCNQ2.

6. The method of claim 1 wherein KCNQ3 is hKCNQ3.

7. The method of claim 1 wherein the compound is an agonist of the potassium current.

8. The method of claim 1 wherein the compound is an antagonist of the potassium current.

9. The method of claim 1 wherein the activity of the potassium channel is measured by a current or a change in membrane voltage, wherein the change in membrane voltage is determined through a voltage sensitive dye.

10. The method of claim 9 wherein the voltage sensitive dye is detectable by fluoresence.

11. The method of claim 1 comprising contacting a compound with a mammalian cell that coexpresses KCNQ2 and KCNQ3, wherein the KCNQ2 and the KCNQ3 form a potassium channel; and measuring the activity of the potassium channel.

12. The method of claim 11 wherein the compound is an agonist of the potassium current.

13. The method of claim 11 wherein the compound is an antagonist of the potassium current.

14. The method of claim 11 wherein the activity of the potassium channel is measured by a current.

15. The method of claim 11 wherein the activity of the potassium channel is measured by a change in membrane voltage wherein the change in membrane voltage is determined through a voltage sensitive dye.

16. The method of claim 15 wherein the voltage sensitive dye is detectable by fluoresence.

17. The method of claim 1 comprising contacting a compound with a mammalian cell that coexpresses hKCNQ2 and hKCNQ3, wherein the hKCNQ2 and the hKCNQ3 form a potassium channel; and measuring the activity of the potassium channel.

18. The method of claim 17 wherein the compound is an agonist of the potassium current.

19. The method of claim 17 wherein the compound is an antagonist of the potassium current.

20. The method of claim 17 wherein the activity of the potassium channel is measured by a current or a change in membrane voltage wherein the change in membrane voltage is determined through a voltage sensitive dye.

21. The method of claim 20 wherein the voltage sensitive dye is detectable by fluoresence.

22. A pharmaceutical composition comprising a pharmaceutically acceptable carrier and a therapeutically effective amount of a compound identified by the screening assay of claim 1 or a pharmaceutically acceptable salt or prodrug form thereof, wherein said compound modulates a potassium channel formed by the coexpression of KCNQ2 and KCNQ3.

23. A method for treating a degenerative neurological disorder involving a potassium channel formed by the coexpression of KCNQ2 and KCNQ3 comprising administering to a host in need of such treatment a therapeutically effective amount of a compound identified by the screening assay of claim 1 or a pharmaceutically acceptable salt or prodrug form thereof.

24. A method for treating epilepsy involving a potassium channel formed by the coexpression of KCNQ2 and KCNQ3 comprising administering to a host in need of such treatment a therapeutically effective amount of a compound identified by the screening assay of claim 1 or a pharmaceutically acceptable salt or prodrug form thereof.


SEQUENCE LISTING (15 sequences)

SEQ ID NO: 1 - DNA, 3232 bp, Homo sapiens
  Unsure (3091)..(3091)
  misc_feature (3091)..(3091): n is a, c, g, or t
gagtgcggaaccgccgcctcggccatgcggctcccggccggggggcctgggctggggcccgcgccgccccccgcgctccgcccccgctgagcctgagcccgacccggggcgcctcccgccaggcaccatggtgcagaagtcgcgcaacggcggcgtataccccggcccgagcggggagaagaagctgaaggtgggcttcgtggggctggaccccggcgcgcccgactccacccgggacggggcgctgctgatcgccggctccgaggcccccaagcgcggcagcatcctcagcaaacctcgcgcgggcggcgcgggcgccgggaagccccccaagcgcaacgccttctaccgcaagctgcagaatttcctctacaacgtgctggagcggccgcgcggctgggcgttcatctaccacgcctacgtgttcctcctggttttctcctgcctcgtgctgtctgtgttttccaccatcaaggagtatgagaagagctcggagggggccctctacatcctggaaatcgtgactatcgtggtgtttggcgtggagtacttcgtgcggatctgggccgcaggctgctgctgccggtaccgtggctggagggggcggctcaagtttgcccggaaaccgttctgtgtgattgacatcatggtgctcatcgcctccattgcggtgctggccgccggctcccagggcaacgtctttgccacatctgcgctccggagcctgcgcttcctgcagattctgcggatgatccgcatggaccggcggggaggcacctggaagctgctgggctctgtggtctatgcccacagcaaggagctggtcactgcctggtacatcggcttcctttgtctcatcctggcctcgttcctggtgtacttggcagagaagggggagaacgaccactttgacacctacgcggatgcactctggtggggcctgatcacgctgaccaccattggctacggggacaagtacccccagacctggaacggcaggctccttgcggcaaccttcaccctcatcggtgtctccttcttcgcgctgcctgcaggcatcttggggtctgggtttgccctgaaggttcaggagcagcacaggcagaagcactttgagaagaggcggaacccggcagcaggcctgatccagtcggcctggagattctacgccaccaacctctcgcgcacagacctgcactccacgtggcagtactacgagcgaacggtcaccgtgcccatgtacagttcgcaaactcaaacctacggggcctccagacttatccccccgctgaaccagctggagctgctgaggaacctcaagagtaaatctggactcgctttcaggaaggaccccccgccggagccgtctccaagtaaaggcagcccgtgcagagggcccctgtgtggatgctgccccggacgctctagccagaaggtcagtttgaaagatcgtgtcttctccagcccccgaggcgtggctgccaaggggaaggggtccccgcaggcccagactgtgaggcggtcacccagcgccgaccagagcctcgaggacagccccagcaaggtgcccaagagctggagcttcggggaccgcagccgggcacgccaggctttccgcatcaagggtgccgcgtcacggcagaactcagaagaagcaagcctccccggagaggacattgtggatgacaagagctgcccctgcgagtttgtgaccgaggacctgaccccgggcctcaaagtcagcatcagagccgtgtgtgtcatgcggttcctggtgtccaagcggaagttcaaggagagcctgcggccctacgacgtgatggacgtcatcgagcagtactcagccggccacctggacatgctgtcccgaattaagagcctgcagtccagagtggaccagatcgtggggcggggcccagcgatcacggacaaggaccgcaccaagggcccggccgaggcggagctgcccgaggaccccagcatgatgggacggctcgggaaggtggagaagcaggtcttgtccatggagaagaagctggacttcctggtgaatatctacatgcagcggatgggcatccccccgacagagaccgaggcctactttggggccaaagagccggagccggcgccgccgtaccacagcccggaagacagccgggagcatgtcgacaggcacggctgcattgtcaagatcgtgcgctccagcagctccacgggccagaagaacttctcggcgcccccggccgcgccccctgtccagtgtccgccctccacctcctggcagccacagagccacccgcgccagggccacggcacctcccccgtgggggaccacggctccctggtgcgcatcccgccgccgcctgcccacgagcggtcgctgtccgcctacggcgggggcaaccgcgccagcatggagttcctgcggcaggaggacaccccgggctgcaggccccccgaggggaacctgcgggacagcgacacgtccatctccatcccgtccgtggaccacgaggagctggagcgttccttcagcggcttcagcatctcccagtccaaggagaacctggatgctctcaacagctgctacgcggccgtggcgccttgtgccaaagtcaggccctacattgcggagggagagtcagacaccgactccgacctctgtaccccgtgcgggcccccgccacgctcggccaccggcgagggtccctttggtgacgtgggctgggccgggcccaggaagtgaggcggcgctgggccagtggacccgcccgcggccctcctcagcacggtgcctccgaggttttgaggcgggaaccctctggggcccttttcttacagtaactgagtgtggcgggaagggtgggccctggaggggcccatgtgggctgaaggatgggggctcctggcagtgaccttttacaaaagttattttccaacaggggctggagggctgggcagggcctgtggctccaggagcagcgtgcaggagcaaggctgccctgtccactctgctcaaggccgcggccgacatcagcccggtgtgaagaggggcggagtgatgacgggtgttgcaacctggcaacaagcngggggttgnccagccganccaagggaagcacanaaggaagctgtnccctaagacctncccnaaaggcggcctgtttggtaagactgcgccttggtccggtgggttccggcagcaaaagcgggttttgccgcccctgtcgtg

SEQ ID NO: 2 - protein, 872 aa, Homo sapiens
  UNSURE (347)..(347): Xaa is unknown
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFXATNLSRTDLHSTWQYYERTVTVPMYSSQTQTYGASRLIPPLNQLELLRNLKSKSGLAFRKDPPPEPSPSKGSPCRGPLCGCCPGRSSQKVSLKDRVFSSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRVDQIVGRGPAITDKDRTKGPAEAELPEDPSMMGRLGKVEKQVLSMEKKLDFLVNIYMQRMGIPPTETEAYFGAKEPEPAPPYHSPEDSREHVDRHGCIVKIVRSSSSTGQKNFSAPPAAPPVQCPPSTSWQPQSHPRQGHGTSPVGDHGSLVRIPPPPAHERSLSAYGGGNRASMEFLRQEDTPGCRPPEGNLRDSDTSISIPSVDHEELERSFSGFSISQSKENLDALNSCYAAVAPCAKVRPYIAEGESDTDSDLCTPCGPPPRSATGEGPFGDVGWAGPRK

SEQ ID NO: 3 - protein, 842 aa, Homo sapiens
  UNSURE (347)..(347)
  misc_feature (347)..(347): Xaa can be any naturally occurring amino acid
MVQKSRNGGVYPGPSGEKKLKVGFVGLDPGAPDSTRDGALLIAGSEAPKRGSILSKPRAGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFXATNLSRTDLHSTWQYYERTVTVPMYSSQTQTYGASRLIPPLNQLELLRNLKSKSGLAFRKDPPPEPSPSPRGVAAKGKGSPQAQTVRRSPSADQSLEDSPSKVPKSWSFGDRSRARQAFRIKGAASRQNSEEASLPGEDIVDDKSCPCEFVTEDLTPGLKVSIRAVCVMRFLVSKRKFKESLRPYDVMDVIEQYSAGHLDMLSRIKSLQSRVDQIVGRGPAITDKDRTKGPAEAELPEDPSMMGRLGKVEKQVLSMEKKLDFLVNIYMQRMGIPPTETEAYFGAKEPEPAPPYHSPEDSREHVDRHGCIVKIVRSSSSTGQKNFSAPPAAPPVQCPPSTSWQPQSHPRQGHGTSPVGDHGSLVRIPPPPAHERSLSAYGGGNRASMEFLRQEDTPGCRPPEGNLRDSDTSISIPSVDHEELERSFSGFSISQSKENLDALNSCYAAVAPCAKVRPYIAEGESDTDSDLCTPCGPPPRSATGEGPFGDVGWAGPRK

SEQ ID NO: 4 - DNA, 2755 bp, Homo sapiens
ggcgacgtggagcaagtcaccttggcgctcggggccggagccgacaaagacgggaccctgctgctggagggcggcggccgcgacgaggggcagcggaggaccccgcagggcatcgggctcctggccaagaccccgctgagccgcccagtcaagagaaacaacgccaagtaccggcgcatccaaactttgatctacgacgccctggagagaccgcggggctgggcgctgctttaccacgcgttggtgttcctgattgtcctggggtgcttgattctggctgtcctgaccacattcaaggagtatgagactgtctcgggagactggcttctgttactggagacatttgctattttcatctttggagccgagtttgctttgaggatctgggctgctggatgttgctgccgatacaaaggctggcggggccgactgaagtttgccaggaagcccctgtgcatgttggacatctttgtgctgattgcctctgtgccagtggttgctgtgggaaaccaaggcaatgttctggccacctccctgcgaagcctgcgcttcctgcagatcctgcgcatgctgcggatggaccggagaggtggcacctggaagcttctgggctcagccatctgtgcccacagcaaagaactcatcacggcctggtacatcggtttcctgacactcatcctttcttcatttcttgtctacctggttgagaaagacgtcccagaggtggatgcacaaggagaggagatgaaagaggagtttgagacctatgcagatgccctgtggtggggcctgatcacactggccaccattggctatggagacaagacacccaaaacgtgggaaggccgtctgattgccgccaccttttccttaattggcgtctccttttttgcccttccagcgggcatcctggggtccgggctggccctcaaggtgcaggagcaacaccgtcagaagcactttgagaaaaggaggaagccagctgctgagctcattcaggctgcctggaggtattatgctaccaaccccaacaggattgacctggtggcgacatggagattttatgaatcagtcgtctcttttcctttcttcaggaaagaacagctggaggcagcatccagccaaaagctgggtctcttggatcgggttcgcctttctaatcctcgtggtagcaatactaaaggaaagctatttacccctctgaatgtagatgccatagaagaaagtccttctaaagaaccaaagcctgttggcttaaacaataaagagcgtttccgcacggccttccgcatgaaagcctacgctttctggcagagttctgaagatgccgggacaggtgaccccatggcggaagacaggggctatgggaatgacttccccatcgaagacatgatccccaccctgaaggccgccatccgagccgtcagaattctacaattccgtctctataaaaaaaaattcaaggagactttgaggccttacgatgtgaaggatgtgattgagcagtattctgccgggcatctcgacatgctttccaggataaagtaccttcagacgagaatagatatgattttcacccctggacctccctccacgccaaaacacaagaagtctcagaaagggtcagcattcaccttcccatcccagcaatctcccaggaatgaaccatatgtagccagaccatccacatcagaaatcgaagaccaaagcatgatggggaagtttgtaaaagttgaaagacaggttcaggacatggggaagaagctggacttcctcgtggatatgcacatgcaacacatggaacggttgcaggtgcaggtcacggagtattacccaaccaagggcacctcctcgccagctgaagcagagaagaaggaggacaacaggtattccgatttgaaaaccatcatctgcaactattctgagacaggccccccggaaccaccctacagcttccaccaggtgaccattgacaaagtcagcccctatgggttttttgcacatgaccctgtgaacctgccccgagggggacccagttctggaaaggttcaggcaactcctccttcctcagcaacaacgtatgtggagaggcccacggtcctgcctatcttgactcttctcgactcccgagtgagctgccactcccaggctgacctgcagggcccctactcggaccgaatctccccccggcagagacgtagcatcacgcgagacagtgacacacctctgtccctgatgtcggtcaaccacgaggagctggagaggtctccaagtggcttcagcatctcccaggacagagatgattatgtgttcggccccaatggggggtcgagctggatgagggagaagcggtacctcgccgagggtgagacggacacagacacggaccccttcacgcccagcggctccatgcctctgtcgtccacaggggatgggatttctgattcagtatggaccccttccaataagcccatttaaaagaggtcactggctgacccctccttgtaatgtagacagactttgtatagttcacttactcttacacccgacgcttaccagcggggacaccaatggctgcatcaaatgcatgcgtgtgcgtggtggccccacccaggcaggggcttcccacagcctcttcctccccatgtcaccacaacaaagtgcttccttttcagcatggtttgcatgactttacactatataaatggttccgctaatctcttctaggataaaaaaaaaaaaaaaaaaaaaaaaa

SEQ ID NO: 5 - protein, 825 aa, Homo sapiens
GDVEQVTLALGAGADKDGTLLLEGGGRDEGQRRTPQGIGLLAKTPLSRPVKRNNAKYRRIQTLIYDALERPRGWALLYHALVFLIVLGCLILAVLTTFKEYETVSGDWLLLLETFAIFIFGAEFALRIWAAGCCCRYKGWRGRLKFARKPLCMLDIFVLIASVPVVAVGNQGNVLATSLRSLRFLQILRMLRMDRRGGTWKLLGSAICAHSKELITAWYIGFLTLILSSFLVYLVEKDVPEVDAQGEEMKEEFETYADALWWGLITLATIGYGDKTPKTWEGRLIAATFSLIGVSFFALPAGILGSGLALKVQEQHRQKHFEKRRKPAAELIQAAWRYYATNPNRIDLVATWRFYESVVSFPFFRKEQLEAASSQKLGLLDRVRLSNPRGSNTKGKLFTPLNVDAIEESPSKEPKPVGLNNKERFRTAFRMKAYAFWQSSEDAGTGDPMAEDRGYGNDFPIEDMIPTLKAAIRAVRILQFRLYKKKFKETLRPYDVKDVIEQYSAGHLDMLSRIKYLQTRIDMIFTPGPPSTPKHKKSQKGSAFTFPSQQSPRNEPYVARPSTSEIEDQSMMGKFVKVERQVQDMGKKLDFLVDMHMQHMERLQVQVTEYYPTKGTSSPAEAEKKEDNRYSDLKTIICNYSETGPPEPPYSFHQVTIDKVSPYGFFAHDPVNLPRGGPSSGKVQATPPSSATTYVERPTVLPILTLLDSRVSCHSQADLQGPYSDRISPRQRRSITRDSDTPLSLMSVNHEELERSPSGFSISQDRDDYVFGPNGGSSWMREKRYLAEGETDTDTDPFTPSGSMPLSSTGDGISDSVWTPSNKPI

SEQ ID NO: 6 - DNA, 2766 bp, Rattus norvegicus
tgactccccatccgacctcccctgccccccgggaggcccgcctttgccttcttttgggggggtgggcggggaggggcgcgcggatcatggcattggagttcccgggcttgcagccgccgccgccgcctcgtccacgtaccccaagcgctccttcttcccggagcagcagcggagaaggcgaagcgcccagtgggggcgaggcagatggggctcaaggctcgcagggcatcgggctcctggcaaagacccccctgagccgtccagttaagaggaacaacgccaagtacaggcgcatccaaactttgatctatgacgccctggagagaccgaggggctgggcgctgctctaccacgcgcttgtgttcctgattgtcctgggatgcttgattctggccgtgctcaccactttcaaggagtatgagactgtgtctggagactggcttttgctgctggaaacatttgctattttcatctttggagctgagtttgctttgaggatctgggctgcaggatgttgctgtcgatacaaaggctggcgtggacggctgaagtttgccaggaagcccctgtgcatgttggatatcttcgtgctgattgcttctgtgccagtggttgccgtgggaaaccagggcaatgtcctggctacctctctgcgaagcctccgcttcctgcagatcctgcgcatgcttcgaatggataggaggggtggcacctggaagctcctgggctcagctatctgtgcccacagcaaagaactcatcaccgcctggtacatcggcttcctgacactcatcctttcttcatttcttgtctacctggtggagaaggatgtgccagagatggatgcccaaggagaggaaatgaaagaggagtttgagacctatgctgatgccctgtggtggggcctgatcacactggccaccattggttatggagacaagacacctaaaacctgggaaggacgtctgattgctgccaccttttctttaatcggcgtctccttttttgctcttccggcaggcatccttggctcaggactggcattgaaggttcaggaacagcaccgtcagaagcactttgagaagagaaggaagccagctgctgaactcatccaggctgcctggagatattatgctaccaaccccaacaggcttgacctggtggcaacctggagattttatgaatcagttgtctctttcccattcttcaggaaagaacaactggaagcagcagccagccaaaagctgggtctcttggatcgggttcgcctttctaatcctcgtggtagcaatactaaaggaaagctatttacccctctgaatgtagatgccatagaagaaagcccttccaaagagccaaagcccgttggcttaaacaataaagagcgtttccgcaccgccttccgcatgaaagcctacgctttctggcagagttctgaagatgccgggacaggagaccccatgacagaagacaggggctatggaaatgacttcctcattgaagacatgatccccaccctaaaggctgccatccgagctgtcagaattctacaattccgtctgtataaaaaaaaattcaaggagacattgaggccttatgatgtaaaagatgtaattgagcagtattccgctggacatcttgacatgctttccaggataaagtaccttcagacaagaatagatatgattttcacccctggacctccatccactccaaaacataagaagtctcagaaagggtcagcatttacctacccatcccagcagtctccaaggaatgaaccatatgtagccagggcagccacatcagaaactgaagaccaaagcatgatggggaagtttgtaaaagttgaaagacaggttcacgacatggggaagaaactggacttcctcgtggacatgcacatgcagcatatggagcgtctgcaggtgcgcgtcaccgagtactacccaacaaagggggcctcctccccagcagaaggggagaagaaagaggacaacaggtattctgacttgaaaaccatcatctgtaactactcagaatcaggcccccccgacccaccctacagcttccaccaggtgcccatcgacagagttggtccctatgggttttttgcacatgaccccgtgaaactgaccagagggggacccagttctacaaaggctcaggccaaccttccctcctcgggaagtacatatgcagagaggcccacagtcctgcccatcttgactcttctggactcatgtgtgagctaccactcccagacagaactgcaaggcccctattcggaccacatctcaccccgccagagacgcagcatcactagggacagtgacacaccactgtccctcatgtccgtcaaccacgaggaactggaacggtctccaagtggcttcagcatctcccaagacagagatgattatgtatttggccccagtgggggttcgagctggatgagggaaaagcggtacctggcagaaggagaaacagacacagatacagaccccttcacgcccagtggatccatgcctatgtcatctactggagatggtatttcagattccatatggaccccttccaacaagcccacttagaaggggtcactggctgactcctggtactgtagtcagactttgtacagctcacttactctcacatctagtgcttaacaatgaggactccagtggctgtgtcaagcgcatgcatgtgcgcggtggcccccctgcaagcaggggcttctcacagccttcttcctccccatgtcac

SEQ ID NO: 7 - protein, 835 aa, Rattus norvegicus
MALEFPGLQPPPPPRPRTPSAPSSRSSSGEGEAPSGGEADGAQGSQGIGLLAKTPLSRPVKRNNAKYRRIQTLIYDALERPRGWALLYHALVFLIVLGCLILAVLTTFKEYETVSGDWLLLLETFAIFIFGAEFALRIWAAGCCCRYKGWRGRLKFARKPLCMLDIFVLIASVPVVAVGNQGNVLATSLRSLRFLQILRMLRMDRRGGTWKLLGSAICAHSKELITAWYIGFLTLILSSFLVYLVEKDVPEMDAQGEEMKEEFETYADALWWGLITLATIGYGDKTPKTWEGRLIAATFSLIGVSFFALPAGILGSGLALKVQEQHRQKHFEKRRKPAAELIQAAWRYYATNPNRLDLVATWRFYESVVSFPFFRKEQLEAAASQKLGLLDRVRLSNPRGSNTKGKLFTPLNVDAIEESPSKEPKPVGLNNKERFRTAFRMKAYAFWQSSEDAGTGDPMTEDRGYGNDFLIEDMIPTLKAAIRAVRILQFRLYKKKFKETLRPYDVKDVIEQYSAGHLDMLSRIKYLQTRIDMIFTPGPPSTPKHKKSQKGSAFTYPSQQSPRNEPYVARAATSETEDQSMMGKFVKVERQVHDMGKKLDFLVDMHMQHMERLQVRVTEYYPTKGASSPAEGEKKEDNRYSDLKTIICNYSESGPPDPPYSFHQVPIDRVGPYGFFAHDPVKLTRGGPSSTKAQANLPSSGSTYAERPTVLPILTLLDSCVSYHSQTELQGPYSDHISPRQRRSITRDSDTPLSLMSVNHEELERSPSGFSISQDRDDYVFGPSGGSSWMREKRYLAEGETDTDTDPFTPSGSMPMSSTGDGISDSIWTPSNKPT

SEQ ID NO: 8 - DNA, 5595 bp, Rattus norvegicus
ctttcatgattggttcagtagaatgcaaaacacctaaggtttttatggcagtagttgataaagttgtggtctgcagaccatccagtttcgatccctttttgtaaataaagctttattaggatagacatttatttacatgttatctgcagctgcttttgttaccacaggatggaacaactgccccagaagctataataaggcttattaaagcctatgcaggtgctgcctggtcttttggttaaaaaaaaagtgtatcatttcctgttttacattaaagatagaattaaatgagaagtctgcttgagatgttttagtttttactattattaaggtttgggtgacttaaccaaaatcataaaaaaaaaggaatactattgttttgtgaacagtgtctgtatattttaagaatatacaagggaaatgtatttaaaaagaaaagagatgaaaaaacaaaagtatacttttctaacattagaacaaaatgaaaatcccgcccatgtaacaaacttggtttcactgaggttctctctgtactaagcaaatgatgtttggaatgttacattgggacaaatggtgcccaagtgatcagacatgtaaagcagacaaatgaaactagcacacaactgtcggtttccaaggagtgttacggtcgccatcttcgcaaagccagacacaaactgcaaccataacaagccgtggtcataaaggcgaaaactgtggcagccacgttgatgttgtactggttatcgttcagtagcggcttcacaaccatcgtagtgttgcattgcaggtcatgcagggaggtggccgctgcttccaataaaaaggcgccgaagtagaagacaaagactacgaaatggtaggcaaaatccagaaagttccagttggcatcaatctgagtcaccataccagagaggaacaaacccaggaagagaagcgaaaagaagaaagctgtcacagatacaaacatgacccatccttgcagcagaggtagaggaacattggaagaagcgaccaaaatccagacaagtcccccaagtacaatctccaggcagacgaaaagcttcccgagtaggtcctcaggatgtcagggccagccggcagggtgatccggggcgccgggaaggacacggcagggttcggggcggcgggactgccgctccgccggccgacatgctgccgctgctacgtctccgcgcaccgccgccaccgtcgccaccgcctcccgcgtcccgcctccgcctcccgggcttggccgccgccgccgccgccgccgccgccgccgctccggttcgcggggttcaggcgtgctgagcgcggaaagggtgtggctacgggccctctgccgacagagccccgccccgtcacgtgagcacaggtgagcgcgcctccgccctggcgcccgtcagggtcaccagcgcaggtgtggtgctccccagccgcagccgcctcggccatgcggctgccggacccggggcctgggctggggcccgcgccaccccctgcgcgccgcccccgctgagcccgcgccggatagggcgccgccggcaccatggtgcaaaagtcgcgcaacggtggcgtgtaccccggcaccagcggggaaaaaaagctcaaggtgggcttcgtggggctggaccccggcgcgcccgactccactcgcgacggcgcgctactcatcgcgggctccgaggcccccaagcgcggcagcgttttgagcaagccgcggacgggcggcgcgggagccgggaagcccccaaagcgcaacgccttctaccgcaagctgcagaatttcctatacaacgtgctagagcggccccgcggttgggcgttcatctaccacgcctacgtgtttcttttagtcttctcctgccttgtgctttccgtgttttccaccatcaaggagtatgagaagagttccgaaggggccctctacatcttggaaatcgtgaccatcgtggtattcggtgttgagtactttgtgagaatctgggctgcaggctgctgctgccggtatcgaggctggaggggccggctcaagtttgccaggaagccattctgtgtgatcgacatcatggtgctgattgcctccattgctgtgctggctgctggctcccagggcaatgtctttgctacgtctgcacttcggagcttgcggttcttacaaatcttacggatgatccgtatggaccggaggggcggcacctggaagctcctgggatcggtggtctacgctcacagcaaggagctggtgactgcgtggtacattggcttcctctgcctcatcctggcctcgtttctggtgtacttggcagaaaagggtgagaatgaccacttcgacacctacgcggatgcactctggtggggtctgatcaccctgacaaccattggctacggggacaagtaccctcagacctggaacgggaggctgttagcagcgacgtttaccctcattggtgtctcattcttcgctcttcctgctggcattttgggatccggctttgccctgaaagtccaagagcagcatcggcaaaaacactttgagaaacggcggaatcctgcggcaggtctgatccagtctgcctggagattctatgctactaacctctcacgcaccgacctgcactccacgtggcagtactacgagcggacagtcactgtccccatgatcagctcacaaactcaaacctatggggcctccagactcattccgcctctgaaccagctggagatgctgaggaatctcaagagcaaatctggactcaccttcaggaaggagccacagccagagccatcaccaagtcagaaggtcagtttgaaagatcgtgtcttctccagcccccgaggcgtggctgccaaggggaaggggtctccccaggcccagacggtccggcggtcccccagtgcggatcagagtctcgatgacagcccaagcaaggtgcccaagagctggagctttggtgaccgcagccgtgcacgccaggctttccgtatcaagggcgctgcatcccggcagaactcagaagaagcaagcctccctggggaggatatcgtggaggacaacaagagctgtaactgcgagtttgtgactgaagatcttacccctggcctcaaagtcagcatcagagctgtgtgtgttatgcggttcttggtatctaagcgaaagttcaaagagagtctgcgcccatatgacgtgatggatgtcatcgaacagtactcggccggacacttggatatgttgtcccgaatcaagagcctgcagtccagagtggaccagattgtggggcggggcccgacaataacggacaaggaccgcaccaaaggcccagcggagacggaactgcccgaagaccccagcatgatgggacgccttgggaaggtggaaaaacaggtcttgtccatggaaaagaagctagacttcctggtgagcatctacacacagagaatgggcatcccaccagcagagacagaggcctatttcggggccaaggagcctgagccggcaccaccctaccacagcccggaggacagccgtgaccatgcagacaagcatggctgtattattaagattgtccgctccaccagctctacgggccagaggaaatacgccgcacccccagtcatgccccctgccgagtgtcccccatccacctcgtggcagcagagccaccagcgccacggcacctcccccgtgggagaccatggctcactggtacgcatcccaccaccccctgcgcacgagcgctcactgtctgcctacagtgggggcaacagagccagtaccgagttcttgaggctggagggcaccccagcctgcaggccctctgaggcagccctgcgggatagcgacacgtccatctccatcccttcggtggaccacgaggagctggagcgttcctttagcggtttcagtatctcccagtccaaggagaacctgaatgccctggccagctgttatgcagctgtggcgccgtgcgccaaggtcaggccctacattgcagagggtgagtctgacacagactcagacctctgcacaccgtgtgggccacccccacgctctgccactggtgaaggcccctttggagatgtggcttgggcagggcctaggaagtgattctgggttgggctgctggccccatgccacacccactcttgttcagttttagagctggagttccagggcctttcttaaagtgacagagcggcatagagtagtgtgggttgtgaggatgctcatgggatctcgctctcagggtcaatgtggagtggaaatgaggcagggttccttagcacatacagtaacattttatagaagttcttttccaaccaggagagggtggttgaggccgggccctgtaggcccttgggagctccctatggcagcaaagctagccctgcctagtcttcttgggggacatattgccctgtgagtgaggagggacagcgtgatggttagcctctgtgagtgggggtgtggctgggagcaagtccgtgggaggtagtggtgaaatgaaacaggcccgtctactgctgatagccagtcctgaggcccatgggtctctcagtttagtctgggagctcaggggtatagtaagtcactggaaacggcccactctcacacctgactgcttatccctccatcccgactgcccccagtaaagcattaccagacccactgttgggtgtggctgggcaatgcccctcatccctgggtaacctagctaaagagctgccacaatcttcctccctcttccaggttatgtggatctttccatgggaaccatctttaggtccttctcctggagctgaggaaggagtgaggcctcagggaccaatctggaacttagaattgatccttagactctcttgacttccacctcttaggggaagactcaactcacagccttttctgaaagggtttcttcagcaggttctggctgcctcgtgccaggtactgtagcccagtaggtatgagtcgacacttcaaggcctctgctctttcctggtgtggcactggcctccaggctgtggccagcactttaagagaatcaccagataagcagaccagccttagccagggcacctccttgcctgctgcccacttggctatacggattcaggcagggctaaatgacatgacgtcttcaaactgccggctctctctacacgagagcatggcctttgagcctggtcaaggatccttcccaatgaagctggccatccagtcctttatccaatagtaggtccctggtctgtggcccctgggtgtttgaggaattgggaacaccttggcctctactctggtgaggaataatccctgtccatcctgtgagtgggcatcttgggccatcagcagctgactttagagggaaacatggtagatataatagagccctcggttgtggccccataatatctggcccagaggtgcctggcattaaatgataacattttggggtgggggccctaaaccctcttgccctcagttttcctggtcactgaggacaagcgctacagagcttgtgcttgggcagagctttatttacttcttccacccttttgaaatgtgtgttctggcaggggtagaggcaacttggg

SEQ ID NO: 9 - protein, 852 aa, Rattus norvegicus
MVQKSRNGGVYPGTSGEKKLKVGFVGLDPGAPDSTRDGALLIAGSEAPKRGSVLSKPRTGGAGAGKPPKRNAFYRKLQNFLYNVLERPRGWAFIYHAYVFLLVFSCLVLSVFSTIKEYEKSSEGALYILEIVTIVVFGVEYFVRIWAAGCCCRYRGWRGRLKFARKPFCVIDIMVLIASIAVLAAGSQGNVFATSALRSLRFLQILRMIRMDRRGGTWKLLGSVVYAHSKELVTAWYIGFLCLILASFLVYLAEKGENDHFDTYADALWWGLITLTTIGYGDKYPQTWNGRLLAATFTLIGVSFFALPAGILGSGFALKVQEQHRQKHFEKRRNPAAGLIQSAWRFYATNLSRTDLHSTWQYYERTVTVPMISSQTQTYGASRLIPPLNQLEMLRNLKSKSGLTFRKEPQPEPSPSQKVSLKDRVFSSPRGVAAKGKGSPQAQTVRRSPSADQSLDDSPSKVPKSWSFGDRSRARQAFRIKGAASRQNSEEASLPGEDIVEDNKSCNCEFVTEDLTPGLKVSIRAVCVMRFLVSKRKFKESLRPYDVMDVIEQYSAGHLDMLSRIKSLQSRVDQIVGRGPTITDKDRTKGPAETELPEDPSMMGRLGKVEKQVLSMEKKLDFLVSIYTQRMGIPPAETEAYFGAKEPEPAPPYHSPEDSRDHADKHGCIIKIVRSTSSTGQRKYAAPPVMPPAECPPSTSWQQSHQRHGTSPVGDHGSLVRIPPPPAHERSLSAYSGGNRASTEFLRLEGTPACRPSEAALRDSDTSISIPSVDHEELERSFSGFSISQSKENLNALASCYAAVAPCAKVRPYIAEGESDTDSDLCTPCGPPPRSATGEGPFGDVAWAGPRK

SEQ ID NO: 10 - DNA, 16 bp, Homo sapiens
ccccgctgagcctgag

SEQ ID NO: 11 - DNA, 20 bp, Homo sapiens
tgtaaaaggtcactgccagg

SEQ ID NO: 12 - DNA, 19 bp, Rattus norvegicus
ttgactccccatccgacct

SEQ ID NO: 13 - DNA, 19 bp, Rattus norvegicus
gcctttgccttcttttggg

SEQ ID NO: 14 - DNA, 16 bp, Rattus norvegicus
accgcgcacatgcatg

SEQ ID NO: 15 - DNA, 19 bp, Rattus norvegicus
gtgacatggggaggaagaa